(19) United States
(12) Patent Application Publication
(10) Pub. No.: US 2010/0015642 A1
(43) Pub. Date: Jan. 21, 2010

(54) B7-H1 AND SURVIVIN IN CANCER

(76) Inventors: Eugene D. Kwon, Rochester, MN (US); John C. Cheville, Pine Island, MN (US); Amy Krambeck, Rochester, MN (US); Susan Kuntz, Rochester, MN (US); Robert H. Thompson, Rochester, MN (US); Haidong Dong, Rochester, MN (US); Christine M. Lohse, Rochester, MN (US); Alexander S. Parker, Jacksonville, FL (US)

Correspondence Address: FISH & RICHARDSON P.C., PO BOX 1022, MINNEAPOLIS, MN, 55440-1022, US

(21) Appl. No.: 12/160,017

(22) PCT Filed: Jan. 5, 2007
(86) PCT No.: PCT/US07/60133

§ 371 Date: Feb. 11, 2009

Related U.S. Application Data
(60) Provisional application No. 60/756,906, filed on Jan. 5, 2006.

(51) Int. Cl. G01N 33/574

(52) U.S. Cl. 435/7.23

(57) ABSTRACT

Methods of determining prognosis of a subject with cancer by assessing expression of B7-H1 and survivin in combination.

### CROSS-REFERENCE TO RELATED APPLICATIONS

This application claims the benefit of priority from U.S. Provisional Application Ser. No. 60/756,906, filed Jan. 5, 2006.

### TECHNICAL FIELD

This invention relates to expression of B7-H1 and survivin in biological samples, and more particularly, to using the expression of B7-H1 and survivin in combination to determine the prognosis of a subject with cancer or to determine risk of cancer progression in a subject with cancer.

### BACKGROUND

The incidence of renal cell carcinoma (RCC) has increased steadily over the last three decades, with mortality rates continuing to rise. Jemal et al. (2005) CA Cancer J. Clin. 55, 10-30. To date, the only acceptable treatment for clinically localized RCC is surgical extirpation. Improvements in imaging technology have led to a stage migration and with accompanying surgical advancements, improvements in patient survival have been noted. Pantuck et al. (2001) J. Urol. 166, 1611-1623. The five-year survival of RCC patients, however, is still unacceptably low. This low survival rate reflects the 30% of patients who present with metastatic disease, and another 25-30% of patients who subsequently develop disseminated disease after surgical excision of the primary tumor. Motzer et al. (1996) N. Engl. J. Med. 335, 865-875; and Leibovich et al. (2003) Cancer. 97, 1663-1671. Other treatment modalities for advanced disease such as chemotherapy and radiation have not been shown to be effective. Immunotherapy is the only adjunct therapy available, but less than 10% of patients benefit with durable responses. Fyfe et al. (1995) J. Clin. Oncol. 13, 688-696. Limited therapeutic options have done little to improve the median survival of 6-10 months seen in metastatic disease. Figlin et al. (1997) J. Urol. 158, 740-750. Since a large percent of patients with clinically localized disease subsequently develop metastasis, there is a need for prognostic biomarkers.

### SUMMARY

The present application is based in part on the discovery that expression of B7-H1 and high levels of survivin in tumors can be used as a prognostic biomarker for RCC. As described herein, patients that have tumors expressing B7-H1 and have high levels of survivin are at an increased risk of progression to distant metastases and death, relative to patients having B7-H1-negative tumors and low survivin expression, B7-H1-negative tumors with high survivin expression, or B7-H1-positive tumors with low survivin expression.

In one aspect, the present application features a method of determining the prognosis of a subject with cancer (e.g., renal cell carcinoma). The method includes providing a tissue sample from the subject (e.g., a human); and assessing in the tissue sample the presence or absence of expression of B7-H1 and survivin, wherein the presence of expression of B7-H1 and survivin in the tissue sample indicates the subject is more likely to die of the cancer than if B7-H1 and survivin expression is absent or if B7-H1 or survivin is singly expressed in the tissue sample. Expression can be assessed by detecting the presence or absence of polypeptide. For example, detecting can include contacting the tissue sample with an antibody that binds to B7-H1 and an antibody that binds to survivin. Each antibody can be fluorescently labeled. Detecting also can include fluorescence flow cytometry (FFC) or immunohistochemistry. The tissue sample can be selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. Renal tissue is particularly useful.

The present application also features a method of determining risk of cancer progression in a subject with cancer. The method includes providing a tissue sample from the subject; and assessing in the tissue sample the presence or absence of expression of B7-H1 and survivin, wherein the presence of expression of B7-H1 and survivin in the tissue sample indicates the subject is at more risk of cancer progression than if B7-H1 and survivin expression is absent or if B7-H1 or survivin is singly expressed in the tissue sample. Expression can be assessed by detecting the presence or absence of polypeptide. For example, detecting can include contacting the tissue sample with an antibody that binds to B7-H1 and an antibody that binds to survivin. Each antibody can be fluorescently labeled. Detecting also can include FFC or immunohistochemistry. The tissue sample can be selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. Renal tissue is particularly useful.

In another aspect, the present application features an article of manufacture that includes a first antibody that binds to a B7-H1 polypeptide and a second antibody that binds to a survivin polypeptide. The first antibody can be labeled with a first label and the second antibody can be labeled with a second label, wherein the first and second labels are different. The first and second labels can be fluorescent labels.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention will be apparent from the following description, from the drawings and from the claims.

### DESCRIPTION OF DRAWINGS

FIG. 1 is a graph depicting the association of B7-H1 expression with cancer-specific survival for 298 patients with clear cell RCC. Cancer-specific survival rates (SE, number still at risk) at 1, 5, and 10 years following nephrectomy were 76.2% (5.2%, 50), 39.1% (6.2%, 23), and 33.6% (6.1%, 13), respectively, for patients with B7-H1-positive tumors compared with 94.5% (1.5%, 200), 82.5% (2.7%, 157), and 76.8% (3.0%, 107), respectively, for patients with B7-H1-negative tumors.

FIG. 2 is a graph depicting the association of survivin expression with cancer-specific survival for 298 patients with clear cell RCC. Cancer-specific survival rates (SE, number still at risk) at 1, 5, and 10 years following nephrectomy were 76.5% (4.5%, 66), 40.8% (5.4%, 30), and 36.3% (5.4%, 16), respectively, for patients with high-survivin tumors compared with 96.4% (1.3%, 184), 86.5% (2.5%, 150), and 80.3% (3.0%, 104), respectively, for patients with low-survivin tumors.

FIG. 3 is a graph depicting the association of the combination of B7-H1 and survivin expression with cancer-specific survival for 298 patients with clear cell RCC. Cancer-specific survival rates (SE, number still at risk) at 1, 5, and 10 years following nephrectomy were 97.0% (1.3%, 158), 89.3% (2.5%, 132), and 84.2% (3.0%, 93), respectively, for patients with B7-H1 negative and low-survivin tumors (−/−), 86.2% (4.8%, 42), 59.7% (7.2%, 25), and 51.9% (7.6%, 14), respectively, for patients with B17-H1-negative and high-survivin tumors (−/+), 93.0% (4.8%, 26), 70.0% (8.9%, 18), and 57.8% (9.8%, 11), respectively, for patients with B7-H1-positive and low-survivin tumors (+/−), and 63.8% (7.8%, 24), 16.2% (6.3%, 5), and 16.2% (6.3%, 2), respectively, for patients with B7-H1-positive and high-survivin tumors (+/+).

FIG. 4 is a graph depicting the association of the combination of B7-H1 and survivin expression with progression-free survival for 260 patients with clinically localized clear cell RCC. Progression-free survival rates (SE, number still at risk) at 1, 5, and 10 years following nephrectomy were 97.4% (1.3%, 148), 89.8% (2.5%, 123), and 84.4% (3.1%, 85), respectively, for patients with B7-H1 negative and low-survivin tumors (−/−), 87.4% (5.3%, 33), 70.3% (7.6%, 21), and 54.3% (9.2%, 9), respectively, for patients with B7-H1-negative and high-survivin tumors (−/+), 80.8% (7.7%, 20), 68.2% (9.3%, 16), and 59.1% (10.1%, 11), respectively, for patients with B7-H1-positive and low-survivin tumors (+/−), and 57.5% (9.8%, 13), 43.3% (10.3%, 5), and 43.3% (10.3%, 2), respectively, for patients with B7-H1-positive and high-survivin tumors (+/+).

### DETAILED DESCRIPTION

In general, the present application provides methods and materials for determining the prognosis and risk of cancer progression of patients with cancer, based on the expression pattern of B7-H1 and survivin. As used herein, the term “B7-H1” refers to B7-H1 from any mammalian species and the term “hB7-H1” refers to human B7-H1. Further details on B7-H1 polypeptides and nucleic acids are provided in U.S. Pat. No. 6,803,192 and co-pending U.S. application Ser. No. 09/649,108, the disclosures of which are incorporated herein by reference in their entirety. The nucleotide and amino acid sequences of hB7-H1 can be found in GenBank under Accession Nos. AF177937 and AAF25807, respectively. B7-H1 (also known as PD-L1) is a negative regulator of T cell-mediated immunity. See, Dong et al. (1999) Nat. Med. 5, 1365-1369; Dong et al. (2002) Nat. Med. 8, 793-800; and Thompson et al. (2004) Proc. Natl. Acad. Sci. USA 101, 17174-17179. This molecule is constitutively expressed on macrophage-lineage cell surfaces and is expressed in multiple human malignancies. Expression of B7-H1 in normal, non-activated mammalian cells is largely, if not exclusively, limited to macrophage-lineage cells and provides a potential costimulatory signal source for regulation of T cell activation. In contrast, aberrant expression of B7-H1 by tumor cells has been implicated in impairment of T cell function and survival, resulting in defective host antitumoral immunity.

As used herein, the term “survivin” refers to survivin from any mammalian species. Further details on survivin polypeptides and nucleic acids are provided in U.S. Pat. No. 6,943,150, the disclosure of which is incorporated herein by reference in its entirety. The nucleotide and amino acid sequences of human survivin can be found in GenBank under Accession Nos. U75285 and AAC51660, respectively. Survivin directly inhibits caspase-3, caspase-7, and caspase-9 activity and is an inhibitor of apoptosis. While survivin is expressed during embryonic and fetal development, it is not detected in normal adult tissue. Survivin has been detected, however, in many human malignancies, including neuroblastoma, colorectal cancer, breast cancer, lung cancer, esophageal cancer, prostate cancer, and pancreatic cancer. See, Lee et al., (2005) BMC Cancer 5, 127 and Ambrosini et al. (1997) Nat. Med. 3, 917-921.

As described herein, expression of B7-H1 and survivin results in a synergistic effect and as such, patients that have B7-H1 positive (i.e., 5% or more of the tumor cells have detectable levels of B7-H1) and high survivin (2% or more of the tumor cells have detectable levels of survivin) tumors in clear cell RCC are at significant risk of cancer progression and mortality. Furthermore, identification of the synergistic activity of B7-H1 and survivin provides a target for therapeutic intervention.

### Methods of Determining Prognosis or Risk of Cancer Progression

The expression pattern of B7-H1 and survivin in combination can be used to determine the prognosis of patients with cancer, and to determine the risk of cancer progression. In general, the methods provided herein include assessing the expression of B7-H1 and survivin in a tissue sample from a subject and correlating the expression pattern with prognosis or risk of cancer progression. Suitable subjects can be mammals, including, for example, humans, non-human primates such as monkeys, baboons, or chimpanzees, horses, cows (or oxen or bulls), pigs, sheep, goats, cats, rabbits, guinea pigs, hamsters, rats, gerbils, and mice. A “tissue sample” is a sample that contains cells or cellular material. Typically, the tissue sample is from a tumor, e.g., a resection or biopsy of a tumor.

As described herein, patients with B7-H1 positive (i.e., 5% or more of the tumor cells have detectable levels of B7-H1) and high survivin (2% or more of the tumor cells have detectable levels of survivin) tumor expression demonstrated substantially worse 5-year cancer-specific survival rates as compared to patients with tumor expression of either marker alone, even after adjusting for other predictive factors such as tumor stage, grade and Eastern Cooperative Oncology Group (ECOG) performance status. Furthermore, when comparing patients having clinically localized disease (stage pN0/pNX pM0), the 5-year progression free survival was only 43.3% for patients with B7-H1 positive and high survivin tumors. In contrast, the 5-year progression free survival was 89.8% for patients with B7-H1-negative and low survivin tumor expression, 70.3% for B7-H1-negative and high survivin tumor expression, and 68.2% for patients with B7-H1-positive tumors and low survivin tumor expression. As such, prognosis of patients and risk of cancer progression (e.g., to distant metastases) can be determined, at least in part, by assessing the expression pattern of B7-H1 and survivin in combination. Other factors that can be considered include, for example, the overall health of the patient and previous responses to therapy. Furthermore, assessing expression of B7-H1 and survivin can provide valuable clues as to the course of action to be undertaken in treatment of the cancer, since expression of B7-H1 and high levels of survivin indicates a particularly aggressive course of cancer (e.g., high risk of metastatic progression or death).

Since a number of cancers express B7-H1 and survivin, the methods provided herein are applicable to a variety of cancers, including, for example, renal cancer, hematological cancer (e.g., leukemia or lymphoma), neurological cancer, melanoma, breast cancer, lung cancer, head and neck cancer, gastrointestinal cancer, liver cancer, pancreatic cancer, genitourinary cancer, bone cancer, and vascular cancer. As such, suitable tissue samples for assessing B7-H1 and survivin expression can include, for example, lung, epithelial, connective, vascular, muscle, nervous, skeletal, lymphatic, prostate, colon, cervical, breast, spleen, gastric, intestinal, oral, esophageal, dermal, liver, bladder, thyroid, thymic, adrenal, brain, gallbladder, pancreatic, uterine, ovarian, and testicular tissue. For example, renal, breast, colon, esophageal, and nervous tissue samples are particularly useful for determining the prognosis of a patient with RCC, breast cancer, colorectal cancer, esophageal cancer, or neuroblastoma, respectively.

In some embodiments, expression of B7-H1 and survivin can be tested in leukocytes present in any of the above-listed tissues. Leukocytes infiltrating the tissue can be T cells (CD4+ T cells and/or CD8+ T cells) or B lymphocytes. Such leukocytes can also be neutrophils, eosinophils, basophils, monocytes, macrophages, histiocytes, or natural killer cells. As described herein, there is a significant association between the presence of B7-H1 and high survivin expression and the presence of infiltration of the tissues by leukocytes.

Methods of assessing B7-H1 and survivin expression (RNA and/or polypeptide) can be quantitative, semi-quantitative, or qualitative. Thus, in some embodiments, the level of B7-H1 and survivin expression can be determined as a discrete value. For example, where quantitative RT-PCR is used, the level of expression of B7-H1 or survivin mRNA can be measured as a numerical value by correlating the detection signal derived from the quantitative assay to the detection signal of a known concentration of: (a) B7-H1 or survivin nucleic acid sequence (e.g., B7-H1 cDNA or B7-H1 transcript); or (b) a mixture of RNA or DNA that contains a nucleic acid sequence encoding B7-H1 or survivin. Alternatively, the level of B7-H1 or survivin expression can be assessed using any suitable semi-quantitative/qualitative method, including any of a variety of semi-quantitative/qualitative systems known in the art. Thus, the level of expression of B7-H1 or survivin in a cell or tissue sample can be expressed as, for example, (a) one or more of “excellent”, “good”, “satisfactory”, “unsatisfactory”, and/or “poor;” (b) one or more of “very high”, “high”, “average”, “low”, and/or “very low”; or (c) one or more of “++++”, “+++”, “++”, “+”, “+/−”, and/or “−”. Where it is desired, the level of expression of B7-H1 or survivin in tissue from a subject can be expressed relative to the expression of B7-H1 or survivin from (a) a tissue of a subject known not be cancerous (e.g., a contralateral kidney or lung, or an uninvolved lymph node); or (b) a corresponding tissue from one or more other subjects known not to have the cancer of interest, or known not to have any cancer.

Typically, the presence or absence of B7-H1 and survivin expression is determined based on protein expression. As used herein, with respect to B7-H1 and protein expression, the term “presence” indicates that ≧5% of the cells in the tissue sample have detectable levels of B7-H1 and “absence” indicates that <5% of the cells in the tissue sample have detectable levels of B7-H1. With respect to survivin and protein expression, the term “presence” or “high” indicates that ≧2% of the cells in the tissue sample have detectable levels of survivin and the term “absence” or “low” indicates that <2% of the cells have detectable levels of survivin. For example, in immunohistochemistry, the term “presence” or “high” indicates that ≧2% of the total slide area stained positive for the anti-survivin antibody.

Any suitable method for detecting expression of a protein in a tissue sample can be used, including methods known in the art. For example, antibodies that bind to an epitope specific for B7-H1 can be used to assess the presence or absence of B7-H1 expression and antibodies that bind to an epitope specific for survivin can be used to assess for the presence or absence of survivin expression. As used herein, the terms “antibody” or “antibodies” include intact molecules (e.g., polyclonal antibodies, monoclonal antibodies, humanized antibodies, or chimeric antibodies) as well as fragments thereof (e.g., single chain Fv antibody fragments, Fab fragments, and F(ab)₂ fragments) that are capable of binding to an epitopic determinant of B7-H1 or survivin (e.g., hB7-H1 or human survivin). The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen.

Antibody fragments that can bind to B7-H1 or survivin can be generated by any suitable technique, including those known in the art. For example, F(ab′)₂ fragments can be produced by pepsin digestion of the antibody molecule; Fab fragments can be generated by reducing the disulfide bridges of F(ab′)₂ fragments. Alternatively, Fab expression libraries can be constructed. See, for example, Huse et al. (1989) Science, 246, 1275. Once produced, antibodies or fragments thereof are tested for recognition of B7-H1 or survivin by standard immunoassay methods including ELISA techniques, radioimmunoassays, and Western blotting. See, Short Protocols in Molecular Biology, Chapter 11, Green Publishing Associates and John Wiley & Sons, Edited by Ausubel, F. M et al., 1992.

Antibodies having specific binding affinity for B7-H1 or survivin can be produced using, for example, standard methods. See, for example, Dong et al. (2002) Nature Med. 8, 793-800. Anti-survivin antibodies are commercially available, e.g., from Dako, Carpenteria, Calif. In general, a B7-H1 or survivin polypeptide can be recombinantly produced, or can be purified from a biological sample, and used to immunize animals. As used herein, the term “polypeptide” refers to a polypeptide of at least five amino acids in length. To produce a recombinant B7-H1 or survivin polypeptide, a nucleic acid sequence encoding the appropriate polypeptide can be ligated into an expression vector and used to transform a bacterial or eukaryotic host cell. Nucleic acid constructs typically include a regulatory sequence operably linked to a B7-H1 or survivin nucleic acid sequence. Regulatory sequences do not typically encode a gene product, but instead affect the expression of the nucleic acid sequence. In bacterial systems, a strain of Escherichia coli such as BL-21 can be used. Suitable E. coli vectors include the pGEX series of vectors that produce fusion proteins with glutathione S-transferase (GST). Transformed E. coli are typically grown exponentially, then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

Mammalian cell lines that stably express a B7-H1 or survivin polypeptide can be produced by using expression vectors with the appropriate control elements and a selectable marker. For example, the eukaryotic expression vector pcDNA.3.1+ (Invitrogen, San Diego, Calif.) can be used to express a B7-H1 or survivin polypeptide in, for example, COS cells, Chinese hamster ovary (CHO), or HEK293 cells. Following introduction of the expression vector by electroporation, DEAE dextran, or other suitable method, stable cell lines can be selected. Alternatively, B7-H1 or survivin can be transcribed and translated in vitro using wheat germ extract or rabbit reticulocyte lysate.

In eukaryotic host cells, a number of viral-based expression systems can be utilized to express a B7-H1 or survivin polypeptide. A nucleic acid encoding a B7-H1 or survivin polypeptide can be introduced into a SV40, retroviral or vaccinia based viral vector and used to infect host cells. Alternatively, a nucleic acid encoding a B7-H1 or survivin polypeptide can be cloned into, for example, a baculoviral vector and then used to transfect insect cells.

Various host animals can be immunized by injection of the B7-H1 or survivin polypeptide. Host animals include rabbits, chickens, mice, guinea pigs and rats. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a B7-H1 or survivin polypeptide and standard hybridoma technology. In particular, monoclonal antibodies can be obtained by any technique that provides for the production of antibody molecules by continuous cell lines in culture such as described by Kohler et al. [(1975) Nature, 256, 495], the human B-cell hybridoma technique (Kosbor et al. (1983) Immunology Today, 4, 72; Cote et al. (1983) Proc. Natl. Acad. Sci. USA, 80, 2026), and the EBV-hybridoma technique (Cole et al., “Monoclonal Antibodies and Cancer Therapy”, Alan R. Liss, Inc., pp. 77-96 (1983)). Such antibodies can be of any immunoglobulin class including, IgG, IgM, IgE, IgA, IgD, and any subclass thereof. The hybridoma producing the monoclonal antibodies provided herein can be cultivated in vitro and in vivo.

In immunological assays, an antibody having specific binding affinity for B7-H1 or survivin, or a secondary antibody that binds to an antibody having specific binding affinity for B7-H1 or survivin can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, fluorescein-5-isothiocyanate (FITC), PerCP, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin and then detected with avidin or streptavidin labeled with a molecule described above. In embodiments in which antibodies to B7-H1 and survivin are used in combination, the antibodies can be labeled such that each can be distinctly visualized (e.g., by labeling with two different fluorescent moieties). Methods of detecting or quantifying a label depend on the nature of the label, and include methods known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting B7-H1 or survivin can be performed in a variety of known formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Some protein-detecting assays (e.g., ELISA or Western blot) can be applied to lysates of cells, and others (e.g., immunohistological methods or fluorescence flow cytometry) can be applied to histological sections or unlysed cell suspensions.

In other embodiments, the presence or absence of B7-H1 and survivin expression can be determined based on mRNA levels. As used herein with respect to mRNA expression, the term “presence” indicates that the tumor sample contains a significantly increased level of mRNA relative to (a) a tissue of a subject known not be cancerous (e.g., a contralateral kidney or lung, or an uninvolved lymph node); or (b) a corresponding tissue from one or more other subjects known not to have the cancer of interest, or known not to have any cancer. As used herein with respect to mRNA expression, the term “absence” indicates that the tumor sample does not contain a significantly increased level of mRNA relative to (a) a tissue of a subject known not be cancerous; or (b) a corresponding tissue from one or more other subjects known not to have the cancer of interest, or known not to have any cancer.

Methods for detecting an mRNA in a tissue sample can include those known in the art. For example, cells can be lysed and an mRNA in the lysates or in RNA purified or semi-purified from the lysates can be detected by any of a variety of methods including, without limitation, hybridization assays using detectably labeled gene-specific DNA or RNA probes (e.g., Northern Blot assays), and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

### Articles of Manufacture

Antibodies that can bind to a B7-H1 polypeptide (e.g., hB7-H1) and antibodies that can bind to a survivin polypeptide (e.g., human survivin) can be combined with packaging material and sold as a kit for detecting B7-H1 and survivin from biological samples, determining prognosis of a subject with cancer, or determining risk of cancer progression in a subject. Components and methods for producing articles of manufactures are well known. In addition, the articles of manufacture may further include reagents such as secondary antibodies, sterile water, pharmaceutical carriers, buffers, indicator molecules, solid phases (e.g., beads), and/or other useful reagents (e.g., positive and negative controls) for detecting B7-H1 and survivin from biological samples, determining prognosis of a subject with cancer, or determining risk of cancer progression in a subject. The antibodies can be in a container, such as a plastic, polyethylene, polypropylene, ethylene, or propylene vessel that is either a capped tube or a bottle. In some embodiments, the antibodies can be included on a solid phase such as a handheld device for bedside testing. Instructions describing how the various reagents are effective for determining prognosis of a subject with cancer or determining risk of cancer progression also may be included in such kits.

The invention will be further described in the following examples, which do not limit the scope of the invention described in the claims.

### EXAMPLES

### Example 1

### Materials and Methods

Patient Selection Upon approval from the Mayo Clinic Institutional Review Board, 427 patients were identified from the Mayo Clinic Nephrectomy Registry that were treated with radical nephrectomy for unilateral, sporadic ccRCC between 1990 and 1994. From these 427 cases, 298 (69.8%) had paraffin-embedded tumor tissues available for study. The estimated cancer-specific survival rates (standard error [SE], number still at risk) at 5 years for patients with and without tissue available for study were 72.4% (2.7%, 180) and 79.5% (3.7%, 89), respectively (p=0.823; log-rank test).

Clinical Features. The clinical features studied including age, sex, symptoms at presentation, ECOG performance status, tumor thrombus level and type of surgery. Patients with a palpable flank or abdominal mass, discomfort, gross hematuria, acute onset varicocele, or constitutional symptoms including rash, sweats, weight loss, fatigue, early satiety, and anorexia were considered symptomatic at presentation. The level of tumor thrombus was classified based on radiologic exam as described by Neves & Zincke (1987) Br. J. Urol. 59, 390-395.

Pathologic Features: The pathologic features studied included histologic subtype classified according to the Union Internationale Contre le Cancer, American Joint Committee on Cancer, and Heidelberg guidelines, tumor size, perinephric fat invasion, 2002 primary tumor classification, regional lymph node involvement, distant metastases, the 2002 TNM stage groupings, nuclear grade, coagulative tumor necrosis, and sarcomatoid differentiation. The microscopic slides from all specimens were reviewed by a urologic pathologist without knowledge of patient outcome.

Immunohistochemistry. A histotechnologist in the Mayo Clinic Tissue Acquisition and Cellular/Molecular Analysis (TACMA) facility dissected two, five-micron sections from representative paraffin-embedded tissue blocks for each member of the cohort. One slide was stained with 5H1, a mouse anti-human monoclonal antibody specific for B7-H1, and the other slide was stained with anti-survivin antibody (clone 12C4, 1/100 dilution, Dako, Carpenteria, Calif.). Sections were deparaffinized in three changes of xylene and rehydrated in a graded series of alcohols (100%, 95%, then 70% EtOH). Slides were rinsed in distilled water and unmasked using preheated Target Retrieval Solution (DakoCytomation, Glostrup, Denmark) and a Decloaking Chamber (Biocare Medical, Walnut Creek, Calif.) for 40 minutes then cooled in the buffer for 20 minutes followed by a 5 minute rinse in running distilled water. Following unmasking, slides were blocked for endogenous peroxidase for five minutes with a peroxidase blocking solution (DakoCytomation), rinsed in Tris Buffer Saline with 0.1% Tween 20 (TBST) and incubated for 30 minutes with 1.5% normal horse serum in TBST (DakoCytomation). Slides were then rinsed in TBST and blocked for endogenous avidin and biotin using an Avidin/Biotin Blocking kit (Vector laboratories, Burlingame, Calif.). One slide was subsequently incubated overnight at 4° C. with anti-B7-H1 (clone 5H1) at 1:100. This was followed by 30 minutes of incubation with biotinylated horse anti-mouse IgG and Avidin/Biotin Complex (ABC) reagent from a Vectastain Elite ABC kit (Vector Laboratories, Burlingame, Calif.). Slides were then amplified using a Tyramide Signal Amplification (TSA) Biotin System (Perkin-Elmer, Boston, Mass.) and incubated in 3-Amino-9-Ethylcarbazole (AEC) chromogen (Biocare Medical). Irrelevant isotype matched antibodies were used to control for nonspecific staining.

The other slide was incubated for 30 minutes with anti-survivin (clone 12C4, 1/100 dilution). Sections were rinsed with TBST wash buffer and CSAII Biotin-free TSA system (DAKO Cytomation, Carpenteria, Calif.) was applied and incubated as directed by the manufacturer. The slides were rinsed with TBST wash buffer and incubated in liquid DAB Substrate-Chromogen for 5 minutes then counterstained with Modified Schmidt's Hematoxylin for 5 minutes followed by a 3 minute tap water rinse to blue sections, dehydrated, cleared and mounted with a permanent mounting media.

Quantification of B 7-H1 Protein Expression: The percentages of tumor cells that stained positive for B7-H1 were reviewed independently by two urologic pathologists and quantified in 5% increments. The tumor was considered positive for B7-H1 if there was histologic evidence of cell-surface membrane staining. Cases in which <5% of the tumor cells stained were considered negative. When there was a discrepancy in scoring (most commonly 0% versus 5% staining), the cases were reviewed by both pathologists for a consensus using a double-headed microscope. At all times, both pathologists were blinded to clinical outcome.

Quantification of Survivin Protein Expression: A pathologist reviewed the slides stained with anti-survivin and circled the area of greatest staining for further analysis (diameters of 1-4 mm). A cytotechnologist and imaging specialist in the imaging facility scanned the slides using the Bacus Laboratories Inc. Slide Scanner (Bacus Laboratories, Inc.). Computer assisted analysis was performed using the IHC (Immunohistochemistry) Score software (Bacus Laboratories, Inc) to obtain measurements of total area and IHC area. The percentage of total area that stained positive for the anti-survivin antibody was used as an overall measure of survivin expression.

Statistical Methods: Comparisons of B7-H1 and survivin expression by the clinical and pathologic features studied were evaluated using chi-square and Fisher's exact tests. Cancer-specific survival and progression-free survival were estimated using the Kaplan-Meier method. The associations of B7-H1 and survivin expression with outcome were evaluated using Cox proportional hazards regression models univariately, after adjusting for TNM stage, nuclear grade, and ECOG performance status, and after adjusting for the Mayo Clinic SSIGN (TNM Stage, tumor Size, nuclear Grade, and coagulative tumor Necrosis) Score, a composite prognostic score developed specifically for patients with ccRCC (Frank et al. (2002) J. Urol. 168, 2395-2400). Statistical analyses were performed using the SAS software package (SAS Institute; Cary, N.C.). All p-values were two-sided and those <0.05 were considered statistically significant.

### Example 2

### Survival of RCC Patients with Paraffin-Embedded Tissue Samples Available

At last follow-up, 171 of the 298 patients with paraffin-embedded tumor tissues available for study had died; of these 171 patients, 94 died from ccRCC at a median of 2.1 years following nephrectomy (range 0-13). Among the 127 patients who were still alive at last follow-up, the median duration of follow-up was 11.2 years (range 0-15); 102 (80.3%) of these patients had at least 10 years of follow-up. The cancer-specific survival rates (standard error [SE], number still at risk) at 1, 5, and 10 years following nephrectomy were 90.2% (1.8%, 250), 72.4% (2.7%, 180), and 66.7% (2.9%, 120), respectively.

### Example 3

### Quantification of B7-H1 and Survivin Protein Expression

Seventy (23.5%) tumors exhibited aberrant B7-H1 expression. The mean survivin expression level was 2.3% (median 1.1%; range 0.01%-35.8%). High survivin expression (≧2%) was noted in 92 (30.9%) tumors. There were 177 (59.4%) tumors that were B7-H1 negative with low (i.e., ≦2%) survivin expression, 51 (17.1%) that were B7-H1 negative with high survivin expression, 29 (9.7%) that were B7-H1 positive with low survivin expression, and 41 (13.8%) that were B7-H1 positive with high survivin expression. A comparison of clinical and pathologic features by this combination of B7-H1 and survivin expression is shown in Table 1. The combination of B7-H1 and survivin expression was significantly associated with several adverse clinical and pathologic features, with B7-H1 positive/high survivin tumor expression often demonstrating the worst clinical and pathologic profile. For example, almost all (95.1%) of the tumors with B7-H1-positive/high survivin expression were high-grade (i.e., grade 3 or 4) compared with only 39 (22.0%) of the B7-H1-negative/low survivin tumors.

TABLE 1
Comparison of Clinical and Pathologic Features by the Combination of
B7-H1 and Survivin Expression for 298 Patients with Clear Cell RCC

| | Combination of B7-H1 and Survivin Expression | | | | | | | | |
|---|---|---|---|---|---|---|---|---|---|
| | H1 neg/low survivin | | H1 neg/high survivin | | H1 pos/low survivin | | H1 pos/high survivin | | |
| | N = 177 | | N = 51 | | N = 29 | | N = 41 | | |
| Feature | N (%) | | | | | | | | P-value |
| Age at Surgery | | | | | | | | | |
| <65 Years | 92 | (52.0) | 21 | (41.2) | 20 | (69.0) | 17 | (41.5) | 0.065 |
| ≧65 Years | 85 | (48.0) | 30 | (58.8) | 9 | (31.0) | 24 | (58.5) | |
| Sex | | | | | | | | | |
| Female | 71 | (40.1) | 21 | (41.2) | 14 | (48.3) | 15 | (36.6) | 0.800 |
| Male | 106 | (59.9) | 30 | (58.8) | 15 | (51.7) | 26 | (63.4) | |
| Symptoms | | | | | | | | | |
| No | 66 | (37.3) | 20 | (39.2) | 9 | (31.0) | 6 | (14.6) | 0.038 |
| Yes | 111 | (62.7) | 31 | (60.8) | 20 | (69.0) | 35 | (85.4) | |
| Constitutional Symptoms | | | | | | | | | |
| No | 135 | (76.3) | 38 | (74.5) | 20 | (69.0) | 24 | (58.5) | 0.134 |
| Yes | 42 | (23.7) | 13 | (25.5) | 9 | (31.0) | 17 | (41.5) | |
| ECOG Performance Status | | | | | | | | | |
| 0 | 158 | (89.3) | 47 | (92.2) | 26 | (89.7) | 39 | (95.1) | 0.738 |
| ≧1 | 19 | (10.7) | 4 | (7.8) | 3 | (10.3) | 2 | (4.9) | |
| Tumor Thrombus | | | | | | | | | |
| None | 153 | (86.4) | 35 | (68.6) | 20 | (69.0) | 21 | (51.2) | <0.001 |
| Level 0 | 14 | (7.9) | 10 | (19.6) | 3 | (10.3) | 14 | (34.2) | |
| Level 1-IV | 10 | (5.7) | 6 | (11.8) | 6 | (20.7) | 6 | (14.6) | |
| Type of Surgery | | | | | | | | | |
| Nephron-sparing Surgery | 30 | (17.0) | 2 | (3.9) | 6 | (20.7) | 4 | (9.8) | 0.065 |
| Radical Nephrectomy | 147 | (83.0) | 49 | (96.1) | 23 | (79.3) | 37 | (90.2) | |
| Primary Tumor Size | | | | | | | | | |
| <5 cm | 86 | (48.6) | 15 | (29.4) | 11 | (37.9) | 5 | (12.2) | <0.001 |
| 5 to <7 cm | 44 | (24.9) | 11 | (21.6) | 4 | (13.8) | 6 | (14.6) | |
| 7 to <10 cm | 21 | (11.9) | 15 | (29.4) | 8 | (27.6) | 13 | (31.7) | |
| ≧10 cm | 26 | (14.7) | 10 | (19.6) | 6 | (20.7) | 17 | (41.5) | |
| Perinephric Fat Invasion | | | | | | | | | |
| No | 154 | (87.0) | 42 | (82.4) | 21 | (72.4) | 22 | (53.7) | <0.001 |
| Yes | 23 | (13.0) | 9 | (17.6) | 8 | (27.6) | 19 | (46.3) | |
| Primary Tumor | | | | | | | | | |
| Classification | | | | | | | | | |
| pT1a | 71 | (40.1) | 9 | (17.7) | 8 | (27.6) | 2 | (4.9) | <0.001 |
| pT1b | 60 | (33.9) | 16 | (31.4) | 9 | (31.0) | 6 | (14.6) | |
| pT2 | 13 | (7.3) | 5 | (9.8) | 2 | (6.9) | 6 | (14.6) | |
| pT3a | 8 | (4.5) | 5 | (9.8) | 1 | (3.5) | 7 | (17.1) | |
| pT3b | 21 | (11.9) | 15 | (29.4) | 7 | (24.1) | 18 | (43.9) | |
| pT3c | 2 | (1.1) | 0 | (0.0) | 2 | (6.9) | 2 | (4.9) | |
| pT4 | 2 | (1.1) | 1 | (2.0) | 0 | (0.0) | 0 | (0.0) | |
| Regional Lymph Nodes | | | | | | | | | |
| pNX and pN0 | 172 | (97.2) | 48 | (94.1) | 29 | (100.0) | 35 | (85.4) | 0.014 |
| pN1 and pN2 | 5 | (2.8) | 3 | (5.9) | 0 | (0.0) | 6 | (14.6) | |
| Distant Metastases | | | | | | | | | |
| pM0 | 168 | (94.9) | 42 | (82.4) | 26 | (89.7) | 33 | (80.5) | 0.004 |
| pM1 | 9 | (5.1) | 9 | (17.6) | 3 | (10.3) | 8 | (19.5) | |
| TNM Stage Groupings | | | | | | | | | |
| I | 129 | (72.9) | 23 | (45.1) | 15 | (51.7) | 7 | (17.1) | <0.001 |
| II | 12 | (6.8) | 2 | (3.9) | 2 | (6.9) | 3 | (7.3) | |
| III | 24 | (13.6) | 16 | (31.4) | 9 | (31.0) | 21 | (51.2) | |
| IV | 12 | (6.8) | 10 | (19.6) | 3 | (10.3) | 10 | (24.4) | |
| Nuclear Grade | | | | | | | | | |
| 1 | 29 | (16.4) | 0 | (0.0) | 3 | (10.3) | 1 | (2.4) | <0.001 |
| 2 | 109 | (61.6) | 21 | (41.2) | 8 | (27.6) | 1 | (2.4) | |
| 3 | 37 | (20.9) | 22 | (43.1) | 14 | (48.3) | 20 | (48.8) | |
| 4 | 2 | (1.1) | 8 | (15.7) | 4 | (13.8) | 19 | (46.3) | |
| Coagulative Tumor Necrosis | | | | | | | | | |
| No | 157 | (88.7) | 26 | (51.0) | 17 | (58.6) | 5 | (12.2) | <0.001 |
| Yes | 20 | (11.3) | 25 | (49.0) | 12 | (41.4) | 36 | (87.8) | |
| Sarcomatoid Differentiation | | | | | | | | | |
| No | 176 | (99.4) | 48 | (94.1) | 27 | (93.1) | 28 | (68.3) | <0.001 |
| Yes | 1 | (0.6) | 3 | (5.9) | 2 | (6.9) | 13 | (31.7) | |

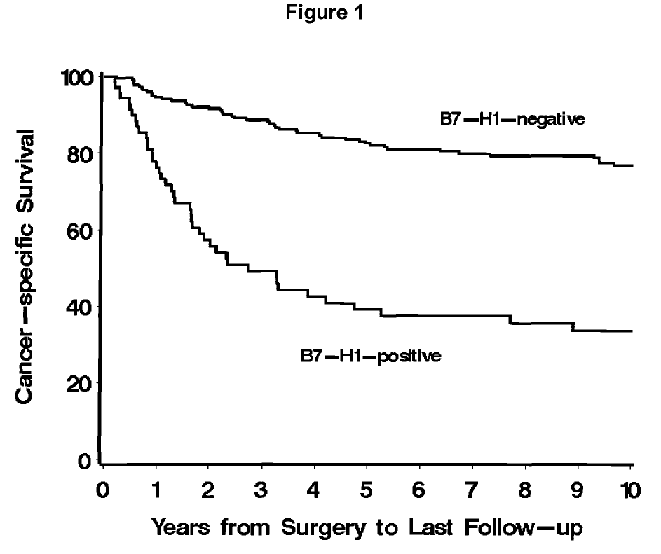

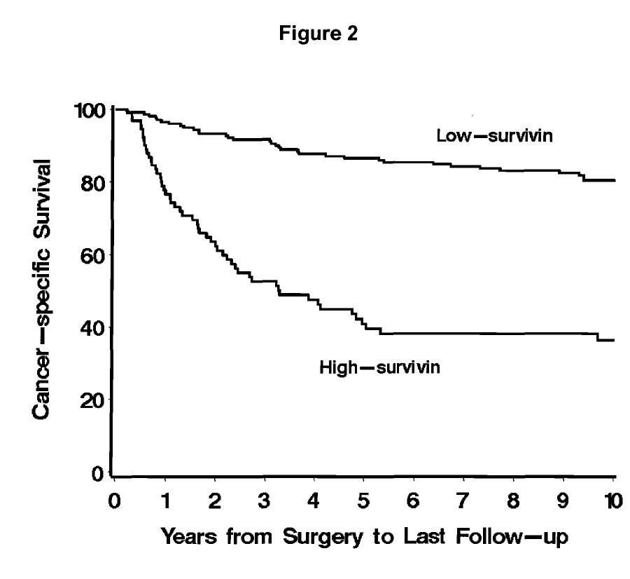

By univariate analysis, patients with a B7-H1-positive tumor were over four times more likely to die from RCC compared with patients with a B7-H1-negative tumor (risk ratio 4.13; 95% CT 2.74-6.22; p<0.001; FIG. 1). The 5-year cancer-specific survival rates were 39.1% for patients with B7-H1-positive tumors compared with 82.5% for patients with B7-H1-negative tumors. Patients with high-survivin tumors were over five times more likely to die from RCC compared with patients with low-survivin tumors (risk ratio 5.14; 95% CT 3.39-7.79; p<0.001; FIG. 2). The 5-year cancer-specific survival rates for patients with high- and low-survivin tumors were 40.8% and 86.5%, respectively.

Stratified analyses did not demonstrate a significant interaction between tumor B7-H1 and survivin expression. For example, among the 228 patients with B7-H1-negative tumors and the 70 patients with B7-H1-positive tumors, the univariate risk ratios for the association of survivin expression with death from RCC were very similar at 4.11 (95% CI 2.38-7.10; p<0.001) and 3.90 (95% CI 1.92-7.91; p<0.001), respectively. Likewise, among the 206 patients with low-survivin tumors and the 92 patients with high-survivin tumors, the univariate risk ratios for the association of B7-H1 expression with death from RCC were 2.89 (95% CI 1.43-5.84; p=0.003) and 3.15 (95% CI 1.82-5.46; p<0.001), respectively. The formal test for interaction between B7-H1 and survivin expression also was not statistically significant (p=0.808). Therefore, the association of B7-H1 and survivin expression with outcome was evaluated after adjusting for each other. When combined together in a model, B7-H1 expression (risk ratio 3.05; 95% CI 2.00-4.67; p<0.001) and survivin expression (risk ratio 4.20; 95% CI 2.73-6.46; p<0.001) were independently significantly associated with death from RCC.

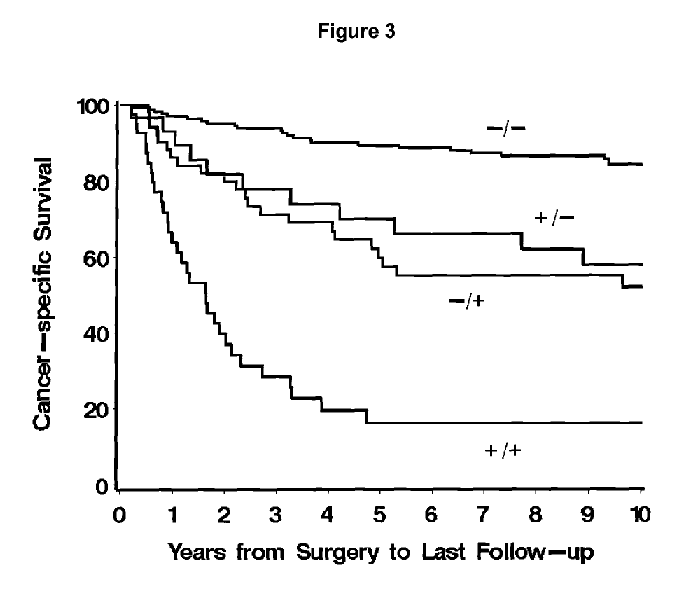

The association of the combination of B7-H1 and survivin expression with cancer-specific survival is illustrated in FIG. 3. The 5-year cancer-specific survival rates for patients with B7-H1-negative/low survivin tumor expression (−/−), B7-H1-negative/high survivin tumor expression (−/+), B7-H1-positive/low survivin tumor expression (+/−), and B7-H1-positive/high survivin tumor expression (+/+) were 89.3%, 59.7%, 70.0%, and 16.2%, respectively. Cancer-specific survival rates did not differ significantly between patients with B7-H1-negative/high survivin tumor expression and those with B7-H I-positive/low survivin tumor expression (p=0.318; log-rank test). The univariate association of this combination variable with death from ccRCC is summarized in Table 2. In a multivariate model, the combination of positive B7-H1/high survivin expression remained significantly associated with death from RCC after adjusting for TNM stage, nuclear grade, and ECOG performance status (risk ratio 3.25; 95% CI 1.77-5.95; p<0.001), and after adjusting for the SSIGN Score (risk ratio 2.81; 95% CI 1.56-5.04; p<0.001; Table 2).

TABLE 2
Association of the Combination of B7-H1 and Survivin Expression
with Death from RCC for 298 Patients with Clear Cell RCC

| Feature | Risk Ratio (95% CI) | | P-value |
|---|---|---|---|
| Univariate Model | | | |
| B7-H1-Negative, Low-Survivin | 1.0 | (reference) | |
| B7-H1-Negative, High-Survivin | 4.03 | (2.34-6.96) | <0.001 |
| B7-H1-Positive, Low-Survivin | 2.85 | (1.41-5.75) | 0.003 |
| B7-H1-Positive, High-Survivin | 12.82 | (7.50-21.92) | <0.001 |
| Adjusted for TNM, Grade, and ECOG | | | |
| B7-H1-Negative, Low-Survivin | 1.0 | (reference) | |
| B7-H1-Negative, High-Survivin | 2.09 | (1.17-3.71) | 0.012 |
| B7-H1-Positive, Low-Survivin | 1.73 | (0.84-3.56) | 0.134 |
| B7-H1-Positive, High-Survivin | 3.25 | (1.77-5.95) | <0.001 |
| Adjusted for SSIGN Score | | | |
| B7-H1-Negative, Low-Survivin | 1.0 | (reference) | |
| B7-H1-Negative, High-Survivin | 1.45 | (0.80-2.64) | 0.223 |
| B7-H1-Positive, Low-Survivin | 2.34 | (1.16-4.73) | 0.018 |
| B7-H1-Positive, High-Survivin | 2.81 | (1.56-5.04) | <0.001 |

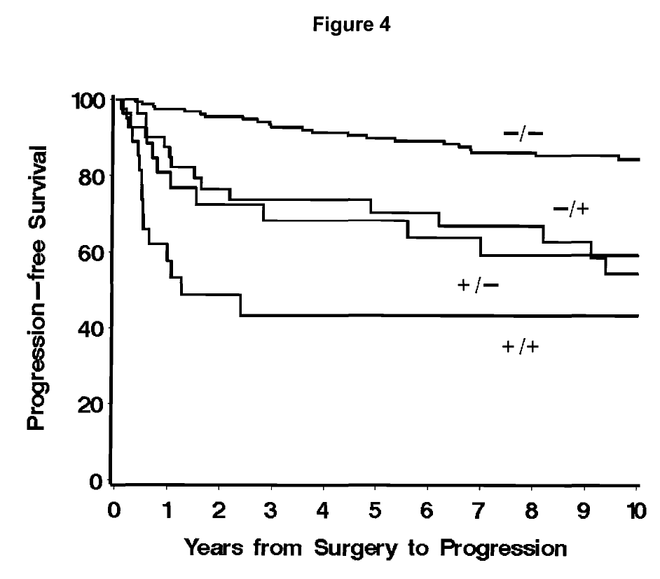

To determine the association of the combination of B7-H1 and survivin expression with cancer progression, a subset of 260 patients was studied with clinically localized (pN0/pNX, pM0) clear cell RCC, of whom 64 (24.6%) progressed to distant metastases at a median of 1.5 years following nephrectomy (range 0-12). The association of the combination of B7-H1 and survivin expression with progression-free survival is illustrated in FIG. 4. The 5-year progression-free survival rates for patients with B7-H1-negative/low survivin tumor expression, B7-H1-negative/high survivin tumor expression, B7-H1-positive tumors/low survivin tumor expression, and B7-H1-positive/high survivin tumor expression were 89.8%, 70.3%, 68.2%, and 43.3%, respectively (p<0.001; log-rank test). Furthermore, in this subset, the combination of positive B7-H1/high survivin expression was significantly associated with death from RCC univariately (risk ratio 13.59; 95% CI 5.98-26.44; p<0.001), after adjusting for TNM stage, nuclear grade, and ECOG performance status (risk ratio 3.32; 95% CI 1.58-6.98; p=0.002), and after adjusting for the SSIGN Score (risk ratio 2.19; 95% CI 1.00-4.78; p=0.050; Table 3).

TABLE 3
Association of the Combination of B7-H1 and Survivin
Expression with Death from RCC for 260 Patients with
Clinically Localized (pNX/pN0, pM0) Clear Cell RCC

| Feature | Risk Ratio (95% CI) | | P-value |
|---|---|---|---|
| Univariate Model | | | |
| B7-H1-Negative, Low-Survivin | 1.0 | (reference) | |
| B7-H1-Negative, High-Survivin | 3.68 | (1.84-7.34) | <0.001 |
| B7-H1-Positive, Low-Survivin | 3.00 | (1.31-6.86) | 0.009 |
| B7-H1-Positive, High-Survivin | 13.59 | (6.98-26.44) | <0.001 |
| Adjusted for TNM, Grade, and ECOG | | | |
| B7-H1-Negative, Low-Survivin | 1.0 | (reference) | |
| B7-H1-Negative, High-Survivin | 2.03 | (0.98-4.23) | 0.058 |
| B7-H1-Positive, Low-Survivin | 1.71 | (0.73-4.01) | 0.216 |
| B7-H1-Positive, High-Survivin | 3.32 | (1.58-6.98) | 0.002 |
| Adjusted for SSIGN Score | | | |
| B7-H1-Negative, Low-Survivin | 1.0 | (reference) | |
| B7-H1-Negative, High-Survivin | 1.34 | (0.62-2.90) | 0.460 |
| B7-H1-Positive, Low-Survivin | 1.97 | (0.86-4.55) | 0.111 |
| B7-H1-Positive, High-Survivin | 2.19 | (1.00-4.78) | 0.050 |

An evaluation of lymphocyte infiltrate also was performed for each tumor. Of those tumors with B7-H1 negative/low survivin tumor expression, only 55/177 (28%) showed lymphocytic infiltration. For the B7-H1 negative/high survivin and B7-H1 positive/low survivin tumors, 31/51 (60.8%) and 21/29 (72.4%) demonstrated lymphocytic infiltration, respectively. The B7-H1 positive/high survivin tumors demonstrated the highest percentage of lymphocytic infiltration 34/41 (82.9%). There was a significant association between combined B7-H1 positive/high survivin and the presence of lymphocytic infiltration (p<0.001; chi-square test).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining the prognosis of a subject with cancer, said method comprising:
(a) providing a tissue sample from said subject; and
(b) assessing in said tissue sample the presence or absence of expression of B7-H1 and survivin, wherein the presence of expression of B7-H1 and survivin in said tissue sample indicates said subject is more likely to die of the cancer than if B7-H1 and survivin expression is absent or B7-H1 or survivin is singly expressed in the tissue sample.

2. The method of claim 1, wherein expression is assessed by detecting the presence or absence of polypeptide.

3. The method of claim 2, wherein detecting comprises contacting said tissue sample with an antibody that binds to B7-H1 and an antibody that binds to survivin.

4. The method of claim 3, wherein each said antibody is fluorescently labeled.

5. The method of claim 2, wherein detecting comprises fluorescence flow cytometry (FFC).

6. The method of claim 2, wherein detecting comprises immunohistochemistry.

7. The method of claim 1, wherein said tissue sample is selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue.

8. The method of claim 7, wherein said tissue sample is renal tissue.

9. The method of claim 1, wherein the cancer is renal cell carcinoma.

10. The method of claim 1, wherein said subject is a human.

11. A method of determining risk of cancer progression in a subject with cancer, said method comprising:
(a) providing a tissue sample from said subject; and
(b) assessing in said tissue sample the presence or absence of expression of B7-H1 and survivin, wherein the presence of expression of B7-H1 and survivin in said tissue sample indicates said subject is at more risk of cancer progression than if B7-H1 and survivin expression is absent or B7-H1 or survivin is singly expressed in the tissue sample.

12. The method of claim 11, wherein expression is assessed by detecting the presence or absence of polypeptide.

13. The method of claim 12, wherein detecting comprises contacting said tissue sample with an antibody that binds to the B7-H1 polypeptide and an antibody that binds to survivin.

14. The method of claim 12, wherein detecting comprises FFC.

15. The method of claim 12, wherein detecting comprises immunohistochemistry.

16. The method of claim 11, wherein said tissue sample is selected from the group consisting of lung, epithelial, connective, vascular, muscle, neural, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue.

17. The method of claim 16, wherein said tissue sample is renal tissue.

18. The method of claim 11, wherein the subject is a human.

19. An article of manufacture, said article of manufacture comprising a first antibody that binds to a B7-H1 polypeptide and a second antibody that binds to a survivin polypeptide.

20. The article of manufacture of claim 19, wherein said first antibody is labeled with a first label and said second antibody is labeled with a second label, wherein said first and second labels are different.

21. The article of manufacture of claim 20, wherein said first and second labels are fluorescent labels.
